(19) United States
(12) Patent Application Publication
(10) Pub. No.: US 2025/0205248 A1
(43) Pub. Date: Jun. 26, 2025

(54) REPURPOSING VARENICLINE AS AN ANTI-INFLAMMATORY AGENT WITH ITS SUPPRESSOR EFFECTS ON INFLAMMATORY CYTOKINES

(71) Applicant: IZMIR EKONOMI UNIVERSITESI, Izmir (TR)

(72) Inventors: Metiner TOSUN, Izmir (TR); Elif BARIS, Izmir (TR)

(73) Assignee: IZMIR EKONOMI UNIVERSITESI, Izmir (TR)

(21) Appl. No.: 18/850,101

(22) PCT Filed: May 15, 2022
(86) PCT No.: PCT/TR2022/050421

§ 371 Date: Sep. 24, 2024

(30) Foreign Application Priority Data
Mar. 24, 2022 (TR) 2022/004561

(51) Int. Cl. A61K 31/55; A61P 29/00

(52) U.S. Cl. CPC A61K 31/55; A61P 29/00

(57) ABSTRACT

A method of a clinical use of varenicline apart from a smoking cessation is provided, including supressing inflammatory cytokines with the varenicline and repositioning the varenicline as an anti-inflammatory drug. The varenicline is repositioned in an LPS-induced in-vitro inflammation model by using a commercially available and widely used immortalized macrophage cell line obtained from male adult Balb/c mice and transformed by Abelson murine leukemia virus.

### CROSS REFERENCE TO THE RELATED APPLICATIONS

This application is the national phase entry of International Application No. PCT/TR2022/050421, filed on May 15, 2022, which is based upon and claims priority to Turkish Patent Application No. 2022/004561, filed on Mar. 24, 2022, the entire contents of which are incorporated herein by reference.

### TECHNICAL FIELD

This invention is related to the method of repurposing the active ingredient of varenicline, which is used in the treatment of smoking cessation, for its use in the treatment of inflammatory diseases with its suppressive activity on inflammatory cytokines.

### BACKGROUND

Sepsis is a systemic inflammatory response that occurs in our body in case of infection. Gram-negative bacterial endotoxin is the best known toxin to initiate inflammation in sepsis, and the lipid A part of the endotoxin in the lipopolysaccharide (LPS) structure is responsible for this effect. This antigenic structure and toxins cause the release of inflammatory cytokines, such as tumor necrosis factor (TNF), interleukin 1 (IL-1), interleukin 6 (IL-6), interleukin 8 (IL-8) and platelet-activating factor (PAF), from the circulating mononuclear phagocytic cells by activating “Toll-like” receptors (TLRs) located on these cells. IL-1 and IL-6 activate T cells and cause the release of other cytokines (IL-2, IL-4) and granulocyte-macrophage colony-stimulating factor (GM-CSF) into the environment. The release of TNFα, interleukins, arachidonic acid metabolites, and other proinflammatory cytokines and their release into the circulation generate the inflammatory response, which also causes septic shock, sepsis, and multi-organ failure.

The inflammatory response resulting from increased inflammatory cytokine release can be controlled by the cholinergic anti-inflammatory pathway mediated by acetylcholine (ACh) released from vagus nerve endings. This effect is defined as an endogenous mechanism that inhibits proinflammatory cytokine release via cholinergic receptors. It has been shown that α7 nicotinic acetylcholine receptors (α7nAChR), expressed on macrophages and other immune response cells, are required for communication between cholinergic nerves and the immune response system. This process is also called the nicotinic anti-inflammatory pathway, since ACh released from T cells in inflamed tissues activates α7nAChRs expressed on macrophages and decreases proinflammatory cytokine release. α7nAChR agonists exert an anti-inflammatory effect by inhibiting the release of TNFα, IL-1, IL-6 and IL-8. It has been reported that the cholinergic anti-inflammatory pathway is stimulated in endotoxemic animals, resulting in increased release of ACh, decreased TNFα levels, and decreased proinflammatory response. Alpha-7nAChR agonist GTS-21 and choline decreased the cytokine levels that increased due to inflammation in sepsis, extended the life span of experimental animals, and had positive effects on tissue damage and correction of multiple organ failure. The fact that ACh and α7nAChR agonist GTS-21 significantly reduces TNFα and HMGB-1 levels in LPS-activated RAW264.7 macrophage cell cultures, and removal of this effect when selective an α7nAChR antagonist is applied, explain the function of α7nAChRs in anti-inflammatory activity. These data suggest that α7nAChR activation may be a potential therapeutic target in the treatment of inflammation. When α7nAChRs on macrophages are activated, inhibition of nuclear translocation of NF-KB and activation of the JAK2/STAT3 signaling pathway are thought to inhibit the transcription of pro-inflammatory cytokines. In the LPS-induced inflammation model in RAW 264.7 macrophage cells, the increase in cytokine levels was shown to be mediated by NF-KB. The fact that choline and nicotine cause NF-KB inhibition and decrease in TNFα levels via α7nAChR activation in RAW 264.7 macrophage cells stimulated with LPS supports these findings.

It is known that in inflammatory conditions, macrophage proliferation and migration increase in parallel with the increase in cytokines. When TLRs are activated by LPS, they induce intracellular signaling cascades in macrophages, increasing cytokine production and activating macrophages. Activated macrophages, on the other hand, migrate to inflamed tissues, encounter pathogenic structures and try to eliminate them by phagocytosis. Various studies have shown the increase in macrophage migration due to the increase in IL-1β, IL-6 and TNFα levels in the LPS-induced inflammation model. In a study examining the function of α7nAChRs in macrophage migration, it was shown that as a result of Ach receptor activation, matrix metalloproteinase-9 (MMP-9) production and macrophage migration were inhibited in LPS-stimulated cells.

Varenicline (Champix®) is a molecule used in the treatment of smoking cessation and has a similar structure to the cytisine alkaloid. Its effectiveness in smoking cessation is achieved through its partial agonistic effect on α4β2nAChRs in the central nervous system. Varenicline has also been shown to be a potent and complete agonist of α7nAChRs. Immunohistochemical analysis showed that varenicline treatment for one week increased the use of damaged forelimbs in animals and decreased inflammation in the corpus striatum in a transient middle cerebral artery ischemia model in mice. In a model of emphysema induced by inhalation of porcine pancreatic elastase (pig pancreatic elastase, PPE) in mice, varenicline administration has been shown to increase alveolar expansion via α7nAChR and reduce inflammation by immunohistochemical analysis. In this study, it was shown that varenicline significantly reduced the number of macrophages, neutrophils and T cells in tissue with the activation of α7nAChR. In a randomized controlled trial in smokers, 3 months of varenicline treatment reduced oxidative stress, atherosclerosis, and endothelial damage. These findings suggested that varenicline might suppress the inflammatory response. There are a limited number of studies conducted in recent years suggesting the anti-inflammatory effect of varenicline with α7nAChR activation.

Although it was reported by the manufacturer (Pfizer) in 1999 that Champix (varenicline 1 and 0.5 mg capsule) may be beneficial in inflammatory bowel diseases, there is no indication approval for this. Varenicline is approved for use only as an adjunct to smoking cessation treatment.

Glucocorticoids (cortisone, prednisone, dexamethasone, etc.), which are widely used in the treatment of inflammatory conditions, are steroid drugs similar to cortisol, an endogenous hormone secreted from the adrenal glands in the body. These are prodrugs and gain effectiveness by transforming into cortisol as a result of enzymatic activity in the body. Cortisol, which has the capacity to activate other sensors (mineralocorticoid receptors) that cause water and sodium retention in the body, is rapidly converted to its inactive (cortisone) form by a critical enzyme (11β-hydroxysteroid dehydrogenase type 2, 11βHSD2) expressed in the liver. Enzyme deficiency or high cortisol levels have caused corticosteroids, which have life-saving efficacy, to be associated with serious problems on the gastrointestinal, musculoskeletal and cardiovascular systems.

### SUMMARY

Here, in the application subject invention, the anti-inflammatory potential of varenicline, which is used in the treatment of smoking cessation, was determined for the first time by suppressing increased cytokine levels in inflammatory conditions. According to the current study, the anti-inflammatory activity of varenicline is not limited to its suppression of cytokine levels, but is also associated with suppressed proliferation and macrophage migration rates (FIGS. 4A-6C).

In the present technique, there is no explanation regarding the technical features of the invention subject to application and the technical effects provided by the invention that is the subject of the application. In current applications, there is no method of using varenicline that provides the potential to have an anti-inflammatory effect.

### Problems Solved by the Invention

The aim of this invention is to repurpose varenicline (which is currently indicated in the treatment of smoking cessation) as an antiinflammatory agent owing to its effectiveness recently discovered in our study.

Another objective of the present invention is to develop a method of use of varenicline that has the potential to exert an anti-inflammatory effect by suppressing the levels of cytokines (14cytokines, three of which are proinflammatory, according to initial observations) increased during inflammation.

### BRIEF DESCRIPTION OF THE DRAWINGS

Figure Legends:

FIGS. 1A-1C. LPS-induced increase in inflammation markers, IL-1β, IL-6, and TNFα, in RAW264.7 cells.

FIGS. 2A-2C. Effects of varenicline on LPS-induced IL-1β, IL-6, and TNFα elevations via nAChR and the comparison with clinically used anti-inflammatory drug dexamethasone. FIGS. 3A-3C. Effects of varenicline on LPS-induced IL-1β, IL-6, and TNFα elevations in the presence or absence of nAChR antagonists

FIGS. 4A-4B. Effects of varenicline on LPS-induced cell proliferation in the presence or absence of nAChR antagonists.

FIGS. 5A-5B. Effects of varenicline on LPS-induced cell migration in the presence or absence of nAChR antagonists

FIGS. 6A-6C. Effects of varenicline on LPS-induced 14 mouse cytokines levels in RAW 264.7 macrophages.

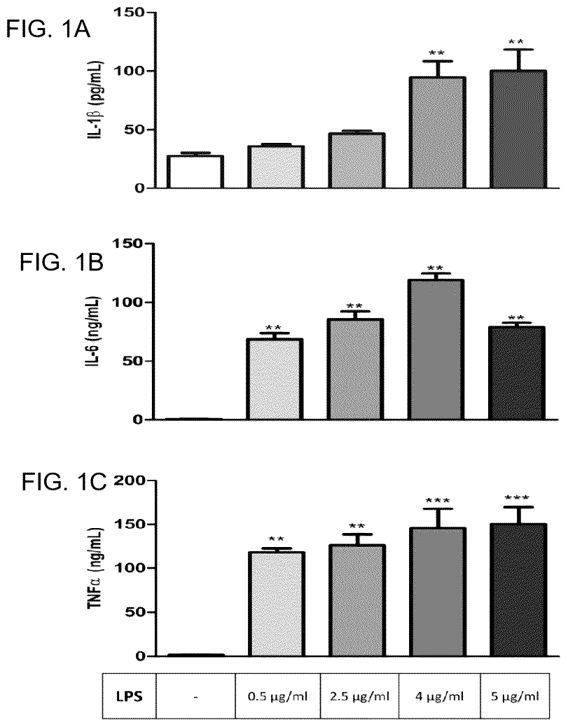

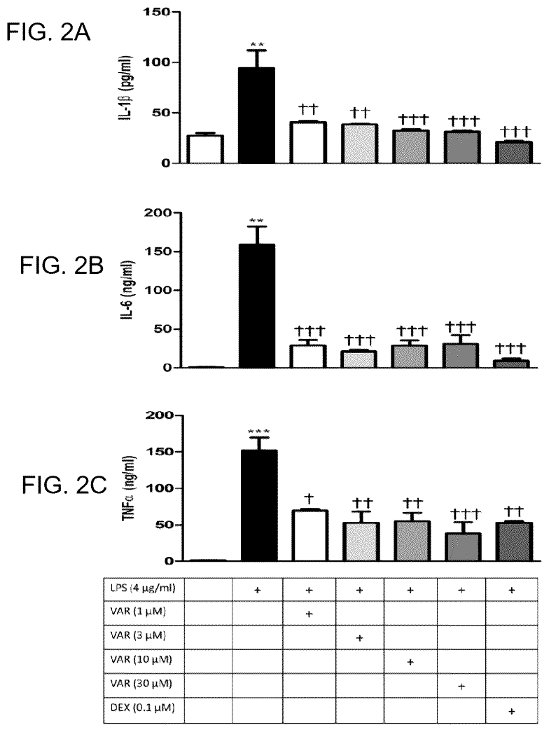

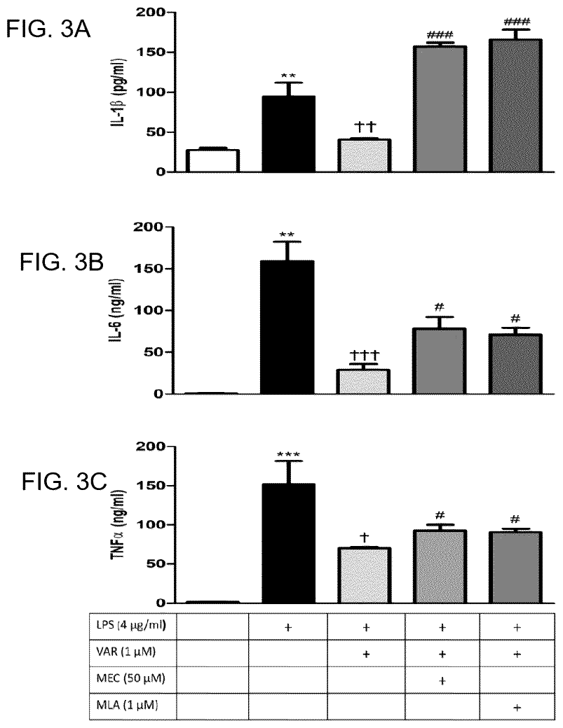

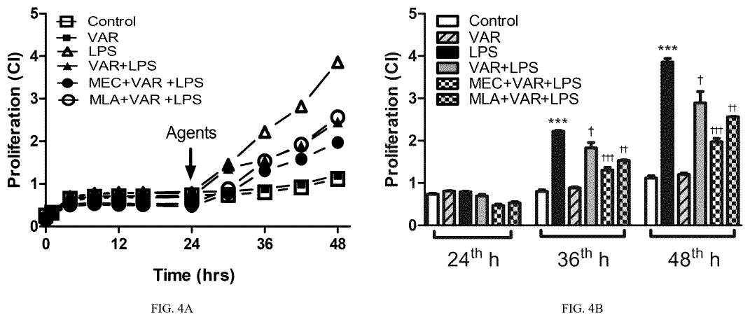

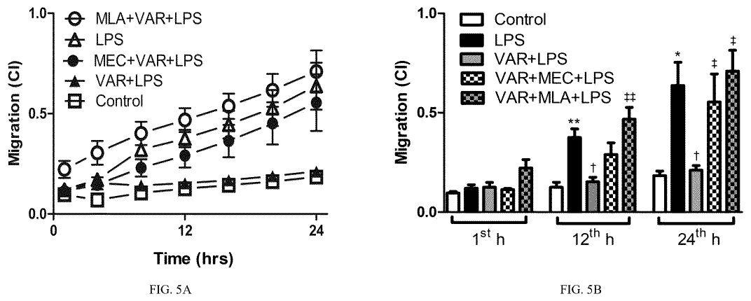

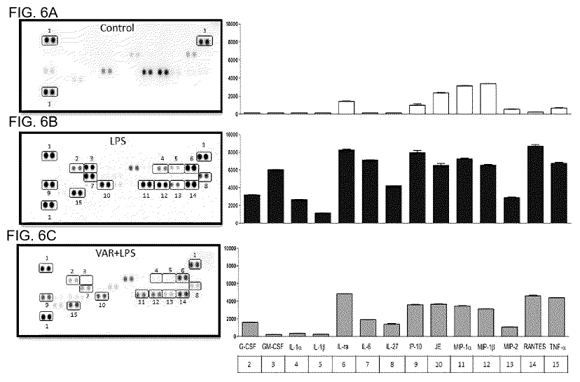

### DETAILED DESCRIPTION OF THE EMBODIMENTS

The invention is for the repurposing of varenicline, which is used in the treatment of smoking cessation, for the treatment of inflammatory diseases and the suppressive effect of varenicline on inflammatory cytokines determined as a part of our study.

This invention points out repurposing of varenicline and provides mechanical insights about the effects of varenicline on α7nAChR-mediated activation of cholinergic anti-inflammatory pathway (CAP), cell proliferation, and migration in the in vitro inflammation model induced by LPS.

RAW 264.7 murine macrophage cells maintained in culture media supplemented with heat inactivated FBS (10%) and penicillin (100 U/ml) and streptomycin (100 μg/ml) at 37° C. in a 5% CO₂ incubator were used during the experimental studies. The cells (500,000/well) were seeded in 48-well culture plates after detachment with scraping incubated for 24 h in serum-free media for reattachment to the surface. In the first group, the cells were treated with LPS at increasing concentrations to determine the effective concentration at which cytokines are released. In the second group, the cells were pretreated with varenicline tartarate with increasing concentrations in presence of LPS to determine effective varenicline concentration on LPS-induced cytokine levels. Additionally, the anti-inflammatory effect of varenicline was compared with that of dexamethasone. In the third group, a non-selective nicotinic ACh receptor antagonist mecamylamine hydrochloride and a selective α7nAChR antagonist methyllycaconitine citrate were applied prior to varenicline and LPS administration in order to investigate the involvement of nicotinic receptors.

The inflammatory response of macrophage cells was evaluated by the change in inflammatory cytokine levels (IL-1β, IL-6 and TNFα) after 24 hours. In other groups where the effect of varenicline will be investigated, the LPS concentration, which triggers the increase in all cytokines determined in the first group and employed for the induction of inflammatory response. Increasing concentrations of varenicline were administered 30 minutes before LPS administration, LPS-induced cytokine levels were analyzed at the end of 24-hour protocol and the effective concentration on cytokine levels were determined in the second group. The effective concentration of varenicline was used in the third group in which the receptor-mediated effects investigated.

The α7nAChR-mediated effect of varenicline was investigated using the specific antagonist methylylcaconitine citrate (MLA) and non-selective nAChR antagonist mecamylamine (MEC) due to varenicline's partial agonistic effect to nAChRs. Antagonist drugs was applied 30 minutes before varenicline and LPS applications. Thus, the role of α7nAChRs and activation of the cholinergic anti-inflammatory pathway was determined at the end of 24-hour protocol by examining LPS-induced inflammatory cytokine levels (IL-1β, IL-6 and TNFα) in addition to 40 different cytokines and chemokines.

The results are as follows for the application of the invention. (1) Cytokine levels (IL-1β, IL-6 and TNFα) examined by the ELISA method were increased at the end of 24-hour protocol after induced by LPS in RAW 264.7 cells. (2) Varenicline administration 30 minutes before the LPS, significantly decreased the levels of all investigated cytokines compared to the LPS group. (3) Vareniklin treatment significantly decreased LPS-induced 14 different cytokines and chemokines including; Granulocyte colony-stimulating factor (G-CSF), Granulocyte-macrophage colony-stimulating factor (GM-CSF), Interleukins (IL-1, IL-6, IL-27), Interleukin-1 receptor antagonist (IL-1ra), Interferon gamma-induced protein 10 (IP-10 or CXCL10), Monocyte chemoattractant protein-1 (MCP-1/CCL2 or JE), Macrophage Inflammatory Protein-1 (MIP-1), regulated upon activation, normal T cell expressed and secreted (RANTES or CCL5), Tumor necrosis factor (TNF α). (4) IL-1β, IL-6, and TNFα levels significantly increased in presence of MEC and MLA groups compared to varenicline-treated groups which indicates nicotinic receptor mediated activation of cholinergic anti-inflammatory pathway (CAP) by varenicline.

What is claimed is:

1. A method of a clinical use of varenicline apart from a smoking cessation, comprising supressing inflammatory cytokines with the varenicline and repositioning the varenicline as an anti-inflammatory drug.

2. The method according to claim 1, wherein the varenicline is repositioned in an LPS-induced in-vitro inflammation model by using a commercially available and widely used immortalized macrophage cell line obtained from male adult Balb/c mice and transformed by Abelson murine leukemia virus.

3. The method according to claim 2, wherein the varenicline is repositioned in RAW 264.7 cells, the RAW 264.7 cells are maintained in a culture medium supplemented with heat-inactivated FBS (10%), 100 U/ml penicillin, and 100 μg/ml streptomycin.

4. The method according to claim 3, wherein the RAW 264.7 cells are maintained at 37° C. in a 5% CO₂ incubator and seeded into 48-well culture plates at an amount of 500,000 cells/well for adherence.

5. The method according to claim 4, wherein a lipopolysaccharide (LPS) is used for producing the LPS-induced in-vitro inflammation model, varenicline tartarate is used as α7nAChR agonist, methylylcaconitine citrate (MLA) is used as selective α7nAChR antagonist, mecamylamine hydrochloride (MEC) is used as non-selective nAChR antagonist, dexamethasone is used as an anti-inflammatory agent.
